(19) United States
(12) Patent Application Publication
(10) Pub. No.: US 2021/0393143 A1
(43) Pub. Date: Dec. 23, 2021

(54) OPTICAL ANKLE-BRACHIAL INDEX AND BLOOD PRESSURE MEASUREMENT SYSTEM AND METHOD

(71) Applicant: MGI, LLC, Ridgefield, NJ (US)

(72) Inventor: Lloyd A. Marks, Westfield, NJ (US)

(21) Appl. No.: 17/353,102

(22) Filed: Jun. 21, 2021

Related U.S. Application Data
(60) Provisional application No. 63/041,425, filed on Jun. 19, 2020.

(51) Int. Cl. A61B 5/022; A61B 5/021; A61B 5/0295; A61B 5/00; A61B 5/1455

(52) U.S. Cl. CPC A61B 5/022; A61B 5/02141; A61B 5/0295; A61B 2562/0233; A61B 5/14551; A61B 5/6829; A61B 2560/0462; A61B 5/6824

(57) ABSTRACT

Optical ankle-brachial index (ABI) and blood pressure measurement systems and methods are disclosed. The blood pressure measurement system and method includes a brachial artery pressure cuff and a reflectance optical pulse detector attached to the pressure cuff. The ABI measurement system and method includes a brachial artery pressure cuff, a first reflectance optical pulse detector attached to the brachial artery pressure cuff, a dorsalis pedis artery pressure cuff or a posterior tibial artery pressure cuff; and a second reflectance optical pulse detector attached to the dorsalis pedis artery pressure cuff or the posterior tibial artery pressure cuff. A computer may also be included in the above systems and methods to detect optical pulses from the cuffs and control the inflation and deflation of the cuffs in order to determine blood pressure and/or ABI.

### CROSS REFERENCE TO RELATED APPLICATION(S)

This application claims priority to U.S. provisional patent application No. 63/041,425, filed on Jun. 19, 2020, which is hereby incorporated herein by reference in its entirety.

### GOVERNMENT SPONSORSHIP

None

### FIELD OF THE INVENTION

Embodiments are in the field of systems and methods for measuring blood pressure and ankle-brachial index (ABI). More particularly, embodiments disclosed herein relate to systems and methods for measuring blood pressure and ABI, including reflectance optical pulse detector(s) attached to pressure cuff(s), which enable simpler, expedient, and accurate procedures which do not have to be performed by a skilled technician.

### BACKGROUND OF THE INVENTION

The ABI is the ratio of the systolic blood pressure in the ankle to the systolic blood pressure in the brachial artery. ABI is an index of peripheral arterial disease (PAD). An ABI ratio between 0.4 and 0.7 means the patient has moderate PAD. An ABI ratio less than 0.4 means they may have severe PAD requiring an intervention. An ABI ratio higher than 1.4 could mean the blood vessels in the limbs are stiff due to advanced age or diabetes. The “gold-standard” method for measuring the ABI is to place a blood pressure cuff over a brachial artery and to place a doppler ultrasound detector/probe, sometimes called a doppler stethoscope, distal to the cuff, for example, over the radial artery. The user listens for the pulse, then inflates the cuff to above systolic pressure, which will make the pulse disappear. The cuff is then deflated until the pulse returns. The reappearance of the pulse occurs at the brachial systolic blood pressure. The ABI can be calculated using one brachial artery measurement or both brachial artery measurements taken from both arms. If both brachial artery systolic pressures are measured, the higher one is used. The process (used to determine the brachial systolic blood pressure) is then repeated with the cuff positioned over each ankle and with the ultrasound probe positioned over the dorsalis pedis or posterior tibial arteries in the foot. The user listens for the pulse using the ultrasound probe, then inflates the ankle cuff to above systolic pressure, which will make the pulse disappear. The ankle cuff is then deflated until the pulse returns. The reappearance of the pulse occurs at the ankle systolic pressure. The ratio of each ankle systolic pressure to the brachial systolic pressure is the ABI for that leg.

That procedure, although a reasonable way to screen for PAD, is time-consuming, requires a skilled technician and, at the current time, is not reimbursed by Medicare as it is considered part of the normal physical exam. As a result, it is rarely done unless the patient has symptoms suggesting PAD such as claudication, which is cramping of the leg muscles when walking. Therefore, people have looked for simpler ways to measure the ABI. Blood pressure (BP) is commonly measured at the present time using machines which use a method call oscillometry. Those machines are very easy to use (they are available as consumer items) and do not have to be performed by a skilled technician. The machines use the pulsations in the cuff pressure as the cuff is inflated and deflated and a computer algorithm to determine the BP from those pulsations. Unfortunately, oscillometry BP measurement is not accurate. It is particularly inaccurate when measuring ankle systolic pressure. Also, as oscillometric BP measuring devices do not provide a true “vascular study” with billable CPT codes, they are not reimbursable. Moreover, oscillometric BP measuring devices are highly inaccurate when measuring diastolic BP.

Thus, it is desirable to provide an ABI and blood pressure measurement system and method that are able to overcome the above disadvantages and which enable simpler, expedient, and accurate procedures which do not have to be performed by a skilled technician.

Advantages of the present invention will become more fully apparent from the detailed description of the invention hereinbelow.

### SUMMARY OF THE INVENTION

Embodiments are directed to a system that measures blood pressure of a patient. The system includes: a pressure cuff configured to be attached to an extremity of the patient; and a reflectance optical pulse detector connected to the pressure cuff and configured to be positioned on a surface of the pressure cuff while facing (i.e., placed on) the extremity of the patient when the pressure cuff is attached to the extremity of the patient.

Embodiments are also directed to a method for measuring blood pressure of a patient. The method includes: providing a reflectance optical pulse detector connected to a pressure cuff; attaching the pressure cuff to an extremity of the patient such that the reflectance optical pulse detector is positioned on a surface of the pressure cuff while facing the extremity of the patient; detecting an optical pulse of the patient using the reflectance optical pulse detector; inflating the pressure cuff until the optical pulse disappears as determined using the reflectance optical pulse detector; deflating the pressure cuff until the optical pulse reappears as determined using the reflectance optical pulse detector; and determining a systolic blood pressure of the patient using the reappearing optical pulse.

Embodiments are further directed to a system that measures ankle-brachial index (ABI) of a patient. The system includes: a brachial artery pressure cuff configured to be attached to an arm of the patient; a first reflectance optical pulse detector connected to the brachial artery pressure cuff and is configured to be positioned on a surface of the brachial artery pressure cuff while facing the patient's brachial artery when the brachial artery pressure cuff is attached to the arm of the patient; a dorsalis pedis artery pressure cuff or a posterior tibial artery pressure cuff, configured to be attached to an ankle of the patient; and a second reflectance optical pulse detector connected to the dorsalis pedis artery pressure cuff or the posterior tibial artery pressure cuff, and is configured to be positioned on a surface of the dorsalis pedis artery pressure cuff or the posterior tibial artery pressure cuff while facing the patient's dorsalis pedis artery or posterior tibial artery when the dorsalis pedis artery pressure cuff or the posterior tibial artery pressure cuff is attached to the ankle of the patient.

Embodiments are yet further directed to a method for measuring ankle-brachial index (ABI) of a patient. The method includes: providing a first reflectance optical pulse detector connected to a brachial artery pressure cuff; attaching the brachial artery pressure cuff to an arm of the patient such that the first reflectance optical pulse detector is positioned on a surface of the brachial artery pressure cuff while facing the patient's brachial artery; providing a second reflectance optical pulse detector connected to a dorsalis pedis artery pressure cuff or a posterior tibial artery pressure cuff; attaching the dorsalis pedis artery pressure cuff or the posterior tibial artery pressure cuff to an ankle of the patient such that the second reflectance optical pulse detector is positioned on a surface of the dorsalis pedis artery pressure cuff or the posterior tibial artery pressure cuff while facing the patient's dorsalis pedis artery or posterior tibial artery detecting a first optical pulse of the patient using the first reflectance optical pulse detector; inflating the brachial artery pressure cuff until the first optical pulse disappears as determined using the first reflectance optical pulse detector; deflating the brachial artery pressure cuff until the first optical pulse reappears as determined using the first reflectance optical pulse detector; and determining a brachial systolic blood pressure of the patient using the reappearing first optical pulse. The reflectance pulse oximeter may be attached to the skin distal to the bladder of the cuff (the optical pulse detector can be positioned within the edge of the cuff distal to the bladder with gentle pressure, perhaps with an elastic band holding it against the skin or on a small tail extending distal to the bladder as long as it is held to the skin by an elastic band or a watch-band like structure.

Additional embodiments and additional features of embodiments for the system that measures blood pressure of a patient, a method for measuring blood pressure of a patient, a system that measures ABI of a patient, and a method for measuring ABI of a patient are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It is understood, however, that the inventive concepts disclosed herein are not limited to the precise arrangements and instrumentalities shown in the figures. The detailed description will refer to the following drawings in which like numerals, where present, refer to like items.

FIG. 1 is a drawing illustrating two brachial artery pressure cuffs attached to arms of a patient and two dorsalis pedis artery pressure cuffs or posterior tibial artery pressure cuffs attached to ankles of the patient;

FIG. 2 is a drawing illustrating an enlarged view of one of the brachial artery pressure cuffs or one of the dorsalis pedis artery pressure cuffs or posterior tibial artery pressure cuffs shown in FIG. 1; and

FIG. 3 is a drawing illustrating an enlarged view of one of the brachial artery pressure cuffs or one of the dorsalis pedis artery pressure cuffs or posterior tibial artery pressure cuffs shown in FIG. 1, along with additional circumferential quadripolar electrodes positioned at proximal and distal ends of the pressure cuff for use with an impedance plethysmograph.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical blood pressure measurement system (or method) or typical ABI measurement system (or method). Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

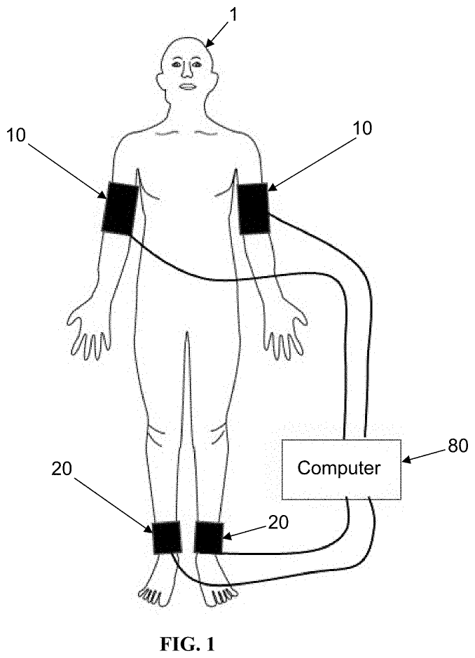

FIG. 1 is a drawing illustrating two brachial artery pressure cuffs 10 attached to arms of a patient 1 and two dorsalis pedis artery pressure cuffs 20 (or posterior tibial artery pressure cuffs) attached to ankles of the patient 1;

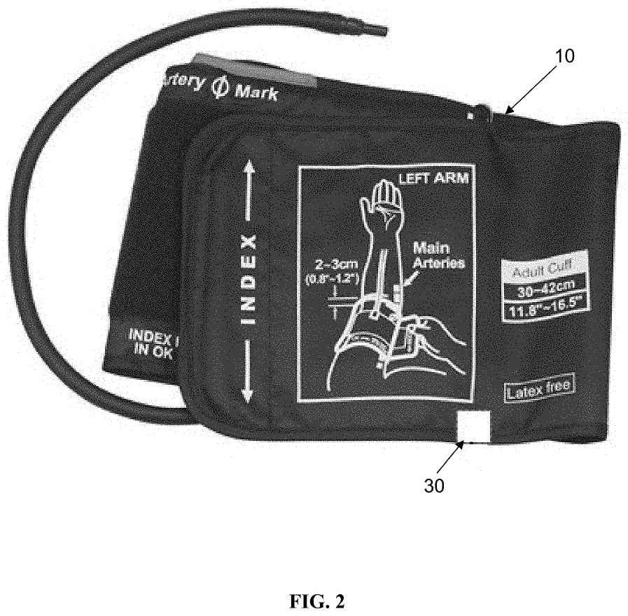

FIG. 2 is a drawing illustrating an enlarged view of one of the brachial artery pressure cuffs 10 or the pressure cuff can alternatively be one of the dorsalis pedis artery pressure cuffs 20 (or posterior tibial artery pressure cuffs) shown in FIG. 1; and

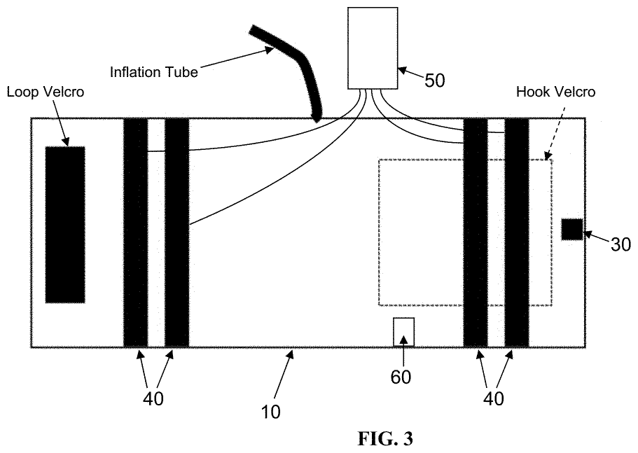

FIG. 3 is a drawing illustrating an enlarged view of one of the brachial artery pressure cuffs 10 or the pressure cuff can alternatively be one of the dorsalis pedis artery pressure cuffs 20 (or posterior tibial artery pressure cuffs) shown in FIG. 1, along with additional circumferential quadripolar electrodes 40 positioned at proximal and distal ends of the pressure cuff 10 for use with an impedance plethysmograph 50.

With reference to FIGS. 1-3, reflectance optical pulse detectors have been developed for use in devices like the Fitbit® and Apple Watch Series 3 and they are reasonably accurate. One company, Rohm, for example makes such a product. An optical signal could also be provided by a laser doppler flowmeter. A laser doppler flowmeter sends out monochromatic (single frequency) laser red light beam which bounces off one or more moving red blood cells causing a doppler shift in the light's frequency. This doppler shifted light is mixed with the outgoing light to produce “beat frequencies” which are the sum and difference of the outgoing a returning light frequencies. The sum of the frequencies is very high and is not used. The difference in the frequencies, however, is low and in the audio range<10 Kz. The “spectral broadening” that is seen in the beat frequencies is an index of skin blood flow although the relationship is not linear. The system includes a series of pressure cuffs 10, 20 (see FIG. 1), one (or two) for the brachial artery(ies) and suitably sized for a patient's arm(s), and one (or two) for the dorsalis pedis artery(ies) or posterior tibial artery(ies) and suitably sized for the patient's ankle(s). Each cuff has a fastening system such as hook and look fasteners (e.g., Velcro® in FIG. 3)) to removable attach the cuffs to the arms and ankles of the patient.

Each cuff has a reflectance optical pulse detector 30 attached/connected to it, preferably on a tail that extends distally from the cuff (i.e., the tail may be considered part of the cuff), from its distal end (such as in FIG. 2) or at or near the middle of a distal edge portion such as in FIG. 3) (but absolutely must be distal to the bladder in the cuff) and aimed downward toward the skin. Some force is required to press the optical detector to the skin but this pressure should preferably not exceed and therefore impede venous outflow which is generally less than 20 mmHg. At least one computer 80 (FIG. 1) is used to first detect the optical pulse in a brachial artery pressure cuff 10. It then inflates the brachial artery pressure cuff 10 (via an inflation tube such as shown in FIGS. 2-3) until the optical pulse disappears. It then deflates the brachial artery pressure cuff 10 (via the inflation tube) until the optical pulse reappears. That determination is done with a computer algorithm (e.g., using a computer 80) such as that described in U.S. Pat. No. 7,887,491. That will occur at the systolic pressure of the brachial artery and has been shown to have similar accuracy as the “gold-standard” doppler ultrasound method described in the BACKGROUND OF THE INVENTION section above.

The process is then repeated for a second brachial artery pressure cuff 10, if present, then each ankle cuff (i.e., the dorsalis pedis artery pressure cuff 20 (or posterior tibial artery pressure cuff). The order in which the cuffs 10, 20 are sampled may be varied and might even be done simultaneously to save time. Thus, systolic pressure will be determined for one (or two) ankles and one (or two) brachial arteries. The computer 80 will then calculate the ABI for each ankle, that is, the ratio of the ankle pressure(s) to the brachial pressure(s). So, if ankle systolic pressures are obtained for both ankles), the ABI for each ankle will be provided to the user or patient on a computer screen/display, in a printout and/or other suitable methods, such as creating computer files with the information so that that information may be later retrieved and reviewed. In other words, the ratio of the ankle systolic pressure to the brachial pressure(s) can be calculated by the computer and displayed to the user. And reports can be saved and printed out. This device would require very little training, unlike an ultrasound technician.

It will be obvious to one skilled in the art, that a database can be created for each patient so that ABI changes can be tracked over time. In addition to providing the ABIs, software may provide a picture of and analyze the shape of the optical waveform, measuring its maximum amplitude, maximum rate of rise and other suitable parameters. A transcutaneous oxygen (TcPO2) sensor 60 could be built into one or more of the pressure cuffs to provide additional information about PAD. A TcPO2 sensor 60 may also provide the optical signal for pulse detection in lieu of an optical plethysmograph and which would clearly allow it to meet Current Procedural Terminology (CPT) Code 93922. A TcPO2 sensor does not measure oxygen saturation, but rather, it measures the concentration of dissolved oxygen in the blood (not in the red blood cells). It produces additional useful information for vascular surgeons such as tissue viability. It's signal is also optical and pulsatile, so it can also be used to detect the reappearance of the blood flow to the tissues when the cuff is deflated. A TcPO2 sensor is not part of an optical plethysmograph although it does use light. It is a different instrument entirely and the addition of one might fill a market niche for vascular surgeons, particularly those performing amputations.

Alternatively, circumferential quadripolar electrodes 40 (see FIG. 3), such as those described in U.S. Pat. Nos. 7,945,318 and 8,019,401, could be built into the proximal and distal ends (or other distanced portions) of the cuffs for use with an impedance plethysmograph 50 as described in U.S. Pat. Nos. 4,548,211 and 10,231,635 (see FIG. 3). It might be advantageous to blow up the cuff slightly, say 10-15 mmHg, when obtaining impedance volume plethysmography measurements to assure good contact of the electrodes with the skin. The addition of volume plethysmography and/or TcPO2 measurements would allow procedures performed with the system to probably meet CPT Code 93922. The use could be widespread as there are many medical indications for performing those studies, particularly for screening for peripheral vascular disease which is present, in some degree, in all men over 50.

This technology could also provide an alternate system and method for measuring systolic and diastolic blood pressure. This disclosure has already described how systolic blood pressure is measured with this technology. As the cuff is deflated, the pulse reappears and is detected optically. That is the systolic blood pressure. As the cuff continues to deflate, the optical pulse will increase in amplitude and rate of rise and plateau. When one or both of those parameters stop increasing, the diastolic blood pressure will have been reached. Thus, the system and method provides an alternate and more accurate way to measure both systolic and diastolic BP than oscillometry which is particularly inaccurate at measuring diastolic blood pressure.

Embodiments are directed to a system that measures the blood pressure of a patient. The system includes: a pressure cuff configured to be attached to an extremity of the patient; and a reflectance optical pulse detector connected to the pressure cuff and configured to be positioned on a surface of the pressure cuff while facing the (e.g., distal end of the) extremity of the patient when the pressure cuff is attached to the extremity of the patient but without any significant pressure applied to it that might interfere with arterial blood flow. Such pressure might be, for example applied with a low tension elastic band. The pressure should not exceed venous pressure which is maximally on the order of 20 mmHg.

In an embodiment, the reflectance optical pulse detector is attached to a distal end of the pressure cuff.

In an embodiment, the pressure cuff is a brachial artery pressure cuff

In an embodiment, the pressure cuff is a dorsalis pedis artery pressure cuff or a posterior tibial artery pressure cuff.

In an embodiment, the system further comprises a laser doppler flowmeter that provides an optical signal for the reflectance optical pulse detector.

In an embodiment, the system may further include a transcutaneous oxygen sensor connected to the pressure cuff.

In an embodiment, the system may further include: an impedance plethysmograph; and circumferential quadripolar electrodes positioned at proximal and distal ends of the pressure cuff. The circumferential quadripolar electrodes are connected to the impedance plethysmograph.

Embodiments are also directed to a method for measuring blood pressure of a patient. The method includes: providing a reflectance optical pulse detector connected to a pressure cuff; attaching the pressure cuff to an extremity of the patient such that the reflectance optical pulse detector is positioned on a (e.g., distal) surface of (or, for example, tail from) the pressure cuff while facing (is placed on) the extremity of the patient (e.g., on an extension of the cuff that extends distally from the distal end of the cuff (so that high pressure in the cuff will not interfere with the measurement of the optical pulse); detecting an optical pulse of the patient using the reflectance optical pulse detector; inflating the pressure cuff until the optical pulse disappears as determined using the reflectance optical pulse detector; deflating the pressure cuff until the optical pulse reappears as determined using the reflectance optical pulse detector; and determining a systolic blood pressure of the patient using the reappearing optical pulse.

In an embodiment, the method may further include: further deflating the pressure cuff until an increase in amplitude of the optical pulse or rate of rise of the optical pulse stops increasing; and determining a diastolic blood pressure of the patient using the further deflating step.

In an embodiment, the pressure cuff is a brachial artery pressure cuff and the systolic blood pressure is a brachial systolic blood pressure.

In an embodiment, the steps of detecting, inflating, deflating, and determining are controlled by at least one computer.

Embodiments are further directed to a system that measures ankle-brachial index (ABI) of a patient. The system includes: a brachial artery pressure cuff configured to be attached to an arm of the patient; a first reflectance optical pulse detector connected to the brachial artery pressure cuff and is configured to be positioned on a surface of the brachial artery pressure cuff (or extension, or tail, thereof that extends distally from the cuff) while facing the patient's brachial artery when the brachial artery pressure cuff is attached to the arm of the patient; a dorsalis pedis artery pressure cuff or a posterior tibial artery pressure cuff, configured to be attached to an ankle of the patient; and a second reflectance optical pulse detector connected to the dorsalis pedis artery pressure cuff or the posterior tibial artery pressure cuff, and is configured to be positioned on a surface of the dorsalis pedis artery pressure cuff or the posterior tibial artery pressure cuff while facing the patient's dorsalis pedis artery or posterior tibial artery when the dorsalis pedis artery pressure cuff or the posterior tibial artery pressure cuff is attached to the ankle of the patient.

In an embodiment of the system that measures ABI, the first reflectance optical pulse detector is attached to a distal end of the brachial artery pressure cuff, distal to the cuff bladder (or a tail that extends from the cuff as long as gentle pressure (as with an elastic band) holds the reflectance pulse oximeter against the skin.

In an embodiment of the system that measures ABI, the system further comprises a laser doppler flowmeter that provides an optical signal for the first reflectance optical pulse detector or the second reflectance optical pulse detector.

Embodiments are yet further directed to a method for measuring ankle-brachial index (ABI) of a patient. The method includes: providing a first reflectance optical pulse detector connected to a brachial artery pressure cuff (or extension, or tail, thereof that extends distally from the cuff); attaching the brachial artery pressure cuff to an arm of the patient such that the first reflectance optical pulse detector is positioned on a surface of the brachial artery pressure cuff while facing the patient's brachial artery; providing a second reflectance optical pulse detector connected to a dorsalis pedis artery pressure cuff or a posterior tibial artery pressure cuff; attaching the dorsalis pedis artery pressure cuff or the posterior tibial artery pressure cuff to an ankle of the patient such that the second reflectance optical pulse detector is positioned on a surface of the dorsalis pedis artery pressure cuff or the posterior tibial artery pressure cuff while facing the patient's dorsalis pedis artery or posterior tibial artery; detecting a first optical pulse of the patient using the first reflectance optical pulse detector; inflating the brachial artery pressure cuff until the first optical pulse disappears as determined using the first reflectance optical pulse detector; deflating the brachial artery pressure cuff until the first optical pulse reappears as determined using the first reflectance optical pulse detector; and determining a brachial systolic blood pressure of the patient using the reappearing first optical pulse.

In an embodiment of the method for measuring ABI, the method may further include: further deflating the brachial artery pressure cuff until an increase in amplitude of the first optical pulse or rate of rise of the first optical pulse stops increasing; and determining a brachial diastolic blood pressure of the patient using the further deflating step.

In an embodiment of the method for measuring ABI, the method may further include: detecting a second optical pulse of the patient using the second reflectance optical pulse detector; inflating the dorsalis pedis artery pressure cuff or the posterior tibial artery pressure cuff until the second optical pulse disappears as determined using the second reflectance optical pulse detector; deflating the dorsalis pedis artery pressure cuff or the posterior tibial artery pressure cuff until the second optical pulse reappears as determined using the second reflectance optical pulse detector; and determining an ankle systolic blood pressure of the patient using the reappearing second optical pulse. In another embodiment, the method may further include: further deflating the dorsalis pedis artery pressure cuff or the posterior tibial artery pressure cuff until an increase in amplitude of the second optical pulse or rate of rise of the second optical pulse stops increasing; and determining an ankle diastolic blood pressure of the patient using the further deflating step.

In an embodiment of the method for measuring ABI, at least one of the first reflectance optical pulse detector and the second reflectance optical pulse detector uses an optical signal from a laser doppler flowmeter.

In an embodiment of the method for measuring ABI, the steps of detecting, inflating, deflating, and determining are controlled by at least one computer.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A system that measures blood pressure of a patient, the system comprising:
a pressure cuff configured to be attached to an extremity of the patient; and
a reflectance optical pulse detector connected to the pressure cuff and configured to be positioned on a surface of the pressure cuff while facing the extremity of the patient when the pressure cuff is attached to the extremity of the patient.

2. The system of claim 1, wherein the reflectance optical pulse detector is attached to a distal end of the pressure cuff.

3. The system of claim 1, wherein the pressure cuff is a brachial artery pressure cuff

4. The system of claim 1, wherein the pressure cuff is a dorsalis pedis artery pressure cuff or a posterior tibial artery pressure cuff.

5. The system of claim 1 further comprising a laser doppler flowmeter that provides an optical signal for the reflectance optical pulse detector.

6. The system of claim 1 further comprising a transcutaneous oxygen sensor connected to the pressure cuff.

7. The system of claim 1 further comprising:
an impedance plethysmograph; and
circumferential quadripolar electrodes positioned at proximal and distal ends of the pressure cuff, wherein the circumferential quadripolar electrodes are connected to the impedance plethysmograph.

8. A method for measuring blood pressure of a patient, the method comprising:
providing a reflectance optical pulse detector connected to a pressure cuff;
attaching the pressure cuff to an extremity of the patient such that the reflectance optical pulse detector is positioned on a surface of the pressure cuff while facing the extremity of the patient;
detecting an optical pulse of the patient using the reflectance optical pulse detector;
inflating the pressure cuff until the optical pulse disappears as determined using the reflectance optical pulse detector;
deflating the pressure cuff until the optical pulse reappears as determined using the reflectance optical pulse detector; and
determining a systolic blood pressure of the patient using the reappearing optical pulse.

9. The method of claim 8 further comprising:
further deflating the pressure cuff until an increase in amplitude of the optical pulse or rate of rise of the optical pulse stops increasing; and
determining a diastolic blood pressure of the patient using the further deflating step.

10. The method of claim 8, wherein the pressure cuff is a brachial artery pressure cuff and the systolic blood pressure is a brachial systolic blood pressure.

11. The method of claim 8, wherein the steps of detecting, inflating, deflating, and determining are controlled by at least one computer.

12. A system that measures ankle-brachial index (ABI) of a patient, the system comprising:
a brachial artery pressure cuff configured to be attached to an arm of the patient;
a first reflectance optical pulse detector connected to the brachial artery pressure cuff and configured to be positioned on a surface of the brachial artery pressure cuff while facing the patient's brachial artery when the brachial artery pressure cuff is attached to the arm of the patient;
a dorsalis pedis artery pressure cuff or a posterior tibial artery pressure cuff, configured to be attached to an ankle of the patient; and
a second reflectance optical pulse detector connected to the dorsalis pedis artery pressure cuff or the posterior tibial artery pressure cuff, and is configured to be positioned on a surface of the dorsalis pedis artery pressure cuff or the posterior tibial artery pressure cuff while facing the patient's dorsalis pedis artery or posterior tibial artery when the dorsalis pedis artery pressure cuff or the posterior tibial artery pressure cuff is attached to the ankle of the patient.

13. The system of claim 12, wherein the first reflectance optical pulse detector is attached to a distal end of the brachial artery pressure cuff.

14. The system of claim 12 further comprising a laser doppler flowmeter that provides an optical signal for the first reflectance optical pulse detector or the second reflectance optical pulse detector.

15. A method for measuring ankle-brachial index (ABI) of a patient, the method comprising:
providing a first reflectance optical pulse detector connected to a brachial artery pressure cuff;
attaching the brachial artery pressure cuff to an arm of the patient such that the first reflectance optical pulse detector is positioned on a surface of the brachial artery pressure cuff while facing the patient's brachial artery;
providing a second reflectance optical pulse detector connected to a dorsalis pedis artery pressure cuff or a posterior tibial artery pressure cuff;
attaching the dorsalis pedis artery pressure cuff or the posterior tibial artery pressure cuff to an ankle of the patient such that the second reflectance optical pulse detector is positioned on a surface of the dorsalis pedis artery pressure cuff or the posterior tibial artery pressure cuff while facing the patient's dorsalis pedis artery or posterior tibial artery;
detecting a first optical pulse of the patient using the first reflectance optical pulse detector;
inflating the brachial artery pressure cuff until the first optical pulse disappears as determined using the first reflectance optical pulse detector;
deflating the brachial artery pressure cuff until the first optical pulse reappears as determined using the first reflectance optical pulse detector; and
determining a brachial systolic blood pressure of the patient using the reappearing first optical pulse.

16. The method of claim 15 further comprising:
further deflating the brachial artery pressure cuff until an increase in amplitude of the first optical pulse or rate of rise of the first optical pulse stops increasing; and
determining a brachial diastolic blood pressure of the patient using the further deflating step.

17. The method of claim 15 further comprising:
detecting a second optical pulse of the patient using the second reflectance optical pulse detector;
inflating the dorsalis pedis artery pressure cuff or the posterior tibial artery pressure cuff until the second optical pulse disappears as determined using the second reflectance optical pulse detector;
deflating the dorsalis pedis artery pressure cuff or the posterior tibial artery pressure cuff until the second optical pulse reappears as determined using the second reflectance optical pulse detector; and
determining an ankle systolic blood pressure of the patient using the reappearing second optical pulse.

18. The method of claim 17 further comprising:
further deflating the dorsalis pedis artery pressure cuff or the posterior tibial artery pressure cuff until an increase in amplitude of the second optical pulse or rate of rise of the second optical pulse stops increasing; and
determining an ankle diastolic blood pressure of the patient using the further deflating step.

19. The method of claim 15, wherein at least one of the first reflectance optical pulse detector and the second reflectance optical pulse detector uses an optical signal from a laser doppler flowmeter.

20. The method of claim 15, wherein the steps of detecting, inflating, deflating, and determining are controlled by at least one computer.
